(19) United States
(12) Patent Application Publication
(10) Pub. No.: US 2010/0047858 A1
(43) Pub. Date: Feb. 25, 2010

(54) HIGH THROUGHPUT SYSTEM FOR CFU ASSAY BY THE USE OF HIGH RESOLUTION DIGITAL IMAGING, DIFFERENTIAL STAINING AND AUTOMATED LABORATORY SYSTEM

(76) Inventors: Maria S. Albano, New York, NY (US); William Rothman, New York, NY (US); Pablo Rubinstein, New York, NY (US)

Correspondence Address: K&L Gates LLP, 1900 MAIN STREET, SUITE 600, IRVINE, CA, 92614-7319, US

(21) Appl. No.: 12/544,999

(22) Filed: Aug. 20, 2009

Related U.S. Application Data
(60) Provisional application No. 61/090,491, filed on Aug. 20, 2008.

(51) Int. Cl. C12Q 1/06; H04N 7/18

(52) U.S. Cl. 435/39; 348/61

(57) ABSTRACT

Disclosed herein is a high throughput system for the objective, standardized determination of colony forming units in populations of hematopoietic cells and indication of successful engraftment. Further disclosed is a laboratory information management system that provides electronic storage of images and data associated with cord blood units.

### CROSS REFERENCE TO RELATED APPLICATIONS

This application claims priority to U.S. Provisional Patent Application No. 61/090,491, filed on Aug. 20, 2008, incorporated herein in its entirety by reference.

### FIELD OF THE INVENTION

The present invention relates to high throughput systems for the objective, standardized determination of colony forming units in populations of hematopoietic cells using a cell detection system and high resolution image analysis.

### BACKGROUND OF THE INVENTION

Umbilical cord blood (CB) is an increasingly accepted graft source for patients lacking related donors. Graft characteristics currently used as determinants of quality and engraftment potential of CB units include the enumeration of total nucleated cells (TNC), CD34+ cells, and colony forming units (CFU). The number of CFU before and after freezing/thawing specimens is a strong independent predictor of CB cell engraftment. Currently, the 14 day CFU assay is the only method that determines the functional state as well as the repopulation capacity and number of hematopoietic progenitor cells. Evaluation of CFU growth as is performed under light microscopy and is time consuming, subjective and difficult to standardize. Further, current methods do not allow for re-counting or visualization of a sample after the assay is complete, as sample plates are discarded at the end of the 14 day assay.

Therefore, a high throughput system to assess CFU number, growth and identify different colony types that is objective and standardized would be desirable. Further, a system that provides positive identification of a sample and storage of an image of the sample and assay results for future reference would be highly desirable.

### SUMMARY OF THE INVENTION

The present disclosure provides a high throughput system for the objective, standardized determination of colony forming units in populations of hematopoietic cells using a cell detection system and high resolution image analysis.

In one embodiment, a standardized colony forming unit assay is provided. The assay comprises the steps of obtaining a sample of a hematopoietic cell-containing tissue or fluid; culturing the cells in a medium that supports growth of hematopoietic precursor cell colonies; staining the colonies; imaging the colonies; counting the colonies for each type of precursor cell; and in certain embodiments, determining the functional state and engraftment potential of the colonies based upon the number of precursor cells and the size of colonies in the sample.

In another embodiment a method for determining the number of hematopoietic precursor cells in a sample is provided. The method comprises obtaining a sample of a hematopoietic cell containing tissue or fluid; culturing the cells in a medium which supports growth of hematopoietic precursor cell colonies; staining the colonies; imaging the colonies; and counting the colonies for each type of precursor cell in the sample.

In another embodiment, an image detection and quantitation system is provided, comprising a high resolution camera, an appropriate light alignment, a broad spectrum flash tube, and optionally, a positive sample ID.

In yet another embodiment, a positive sample identification is provided, comprising a sample and an identification. The sample is a cord blood unit and the identification identifies a specific cord blood unit and information related thereto.

### BRIEF DESCRIPTION OF THE DRAWINGS

FIG. 1 is an image of a 35 mm diameter well comprising unstained (A) and stained (B) CFU (colony forming units) after 14 day cell culture. CFU are identified by the following symbols: ∘ indicates the presence of CFU-GM (granulocyte-macrophage colony forming unit); ⋄ indicates the presence of CFU-E (erythroid colony forming unit); and □ indicates the presence of CFU-GM/E (multilineage colony forming unit). Positive identification is provided on the wells, and includes the testing date/time, CBU ID sample, Dish ID, operator identification, and hood information.

### DETAILED DESCRIPTION OF THE INVENTION

The present disclosure provides a high throughput system for the objective, standardized determination of colony forming units (CFU) in populations of hematopoietic cells using colony staining and high resolution digital imaging . A computer-based laboratory information management system supports the high-throughput CFU assay.

In one embodiment, a standardized colony forming unit assay is provided. The assay comprises the steps of obtaining a sample of a hematopoietic cell containing tissue or fluid; culturing the cells in a medium that supports growth of hematopoietic precursor cell colonies; staining the colonies; imaging the colonies; counting the colonies for each type of precursor cells; and in certain embodiments, determining the functional state and engraftment potential of the colonies based upon the number of precursor cells and size of colonies in the sample.

In another embodiment, a method for determining the number of hematopoietic precursor cells in a sample is provided, which comprises obtaining a sample of a hematopoietic cell containing tissue or fluid; culturing the cells in a medium which supports growth of hematopoietic precursor cell colonies; staining the colonies; imaging the colonies; and counting the colonies for each type of precursor cells in the sample.

The hematopoietic cells can be obtained from any appropriate source, such as blood (peripheral, umbilical cord blood), bone marrow, placenta, etc. In one embodiment, the source is umbilical cord blood. The cells obtained can be cultured in standard media that supports hematopoietic precursor cell colonies for a period of time, such as about 1-21 days, about 5-14 days, or about 10-14 days. In one embodiment, cells are cultured for 14 days. Examples of suitable media include, but are not limited to AMBION® (Invitrogen), STEMLINE® (Sigma), and STEMSPAN®, ALDEFLUOR®, METHOCULT®, and ALDECOUNT® (Stem Cell Technologies). In one embodiment, it is possible to selectively stimulate the proliferation of discrete cell lineages by using appropriate growth factors. Examples of growth factors appropriate for selection of particular cell lineages include, but are not limited to, erythropoietin for red blood cells, thrombopoietin for megakaryocytes, granulocyte stimulating factor (G-CSF) for granulocytes, and granulocyte macrophage stimulating factor (GM-CSF) for granulocyte and macrophage stimulation. In addition, IL-3, IL-6, and stem cell factor, can also be used. These growth factors can be used alone or in various combinations (i.e., cocktails). The estimation of cell numbers after the incubation of a measured number of cells of a given type in separated or unseparated cell samples provides an alternate method of estimating the proliferative capacity of the corresponding cell lineage in the sample. Thus, the acquisition of colony images from whole culture dishes can be expanded to allow numerical estimation of cell numbers and therefore, of actual cell propagation by comparison with the number of cells seeded. In certain embodiments, a subpopulation of CD34+ or light density enriched cells or apheresis product can be isolated before the culturing step.

The colonies are imaged according to one embodiment. Using the disclosed system, CFU can be objectively visualized, differentiated and counted and the digital images can be stored for future review and/or re-classification. The system provides computerized information on optical assay parameters. A combination of high resolution imaging, optional one-step staining, and the traditional CFU assay overcome the technical challenges of the conventional assay. The system supports standardization, classification, and counting reproducibility and high-throughput. One embodiment of an image detection and quantitation system comprises a high resolution camera, appropriate light alignments, a broad spectrum flash tube, and positive sample identification

In one embodiment, a detection system supports the colony forming unit assay. In one embodiment of the detection system, an imaging acquisition system is provided comprising of a full color high speed (for example, 1/125 sec) imaging surface of size comparable to the sample (35 mm diameter dish). The imaging system has a ccd detector (for example, about 39 Megapixel or higher) and results in an image resolution on the order of about 7 microns per pixel. This is then coupled with high distortion-free 1:1 macro focusing lens and a custom designed stage that minimizes stray light, provides reproducible positioning between samples and allows for incorporation of human and machine-readable (i.e., bar code) identification imprinted directly onto each image. The excitation consists of an intense uniform broad spectrum flash (about 1/500 sec) tube. A vibration-free single image is then acquired by a high performance computer for detailed post-imaging analysis, storage and retrospective studies. In one embodiment, the imaging system is an SLR Camera made by Hasselblad and the light source is by Broncolor. However, any suitable camera and light source can be used. The image detection system allows for the acquisition of an image of the CFU assay culture dish where the CFU grown in the culture can be clearly seen with a clear background. An additional advantage of this system is that it allows for a single sample to be imaged on multiple days in exactly the same physical position due to the alignments provided in the disclosed system. Reproducibly aligning the sample for imaging allows an entire well plate or even a single colony to be visualized repeatedly over a period of time. The growth of the colony or colonies can be tracked over time by comparing the images taken on different days. For example, an image of a sample can be compared on day 1, day 5, and day 14. In this way, the growth of a colony of interest or multiple colonies of interest can be monitored.

In one embodiment, the culture is stained. In one embodiment, the culture is stained in a single step. In yet another embodiment, the culture is stained using a multi-step process. Any stain that stains hematopoietic precursor cells can be used. In one embodiment, the stain can be supervital, optical, histochemical, or radiological. The stain should enhance the visual manifestation of the cells lacking hemoglobin, particularly leukocytes. In another embodiment, antibodies specific for markers expressed by cells of selected lineages that are labeled with diverse probes to enhance visualization can be used. In another embodiment, the cells are stained in a single step with MTT (3-[4,5-dimethylthiazol-2y1]-2,5-diphenyltetrazolium bromide), optionally without any wash steps, which allows an even better definition of multilineage colony forming units (CFU-GM/E), erythroid colony forming units (CFU-E), and granulocyte-macrophage colony forming units (CFU-GM) by bestowing a specific color on each type (dark purple, red, and light purple respectively) against a uniformly clear background. Specific colors are generated after a specific incubation time and MTT dilution. For instance, in one embodiment, the incubation time can be about 10-45 min. In another embodiment, the incubation time is about 15-40 min. In yet another embodiment, the incubation time is about 30-40 min. The dilution of the dye can vary according to the desired staining. When MTT is used, a dilution of 1/2 to about 1/10 can be used. In one embodiment, a 1/5 dilution of MTT is used. In one embodiment, staining can be achieved with 30 min incubation at 37° C. followed by 10 min at room temperature, with a 1/5 dilution of MTT.

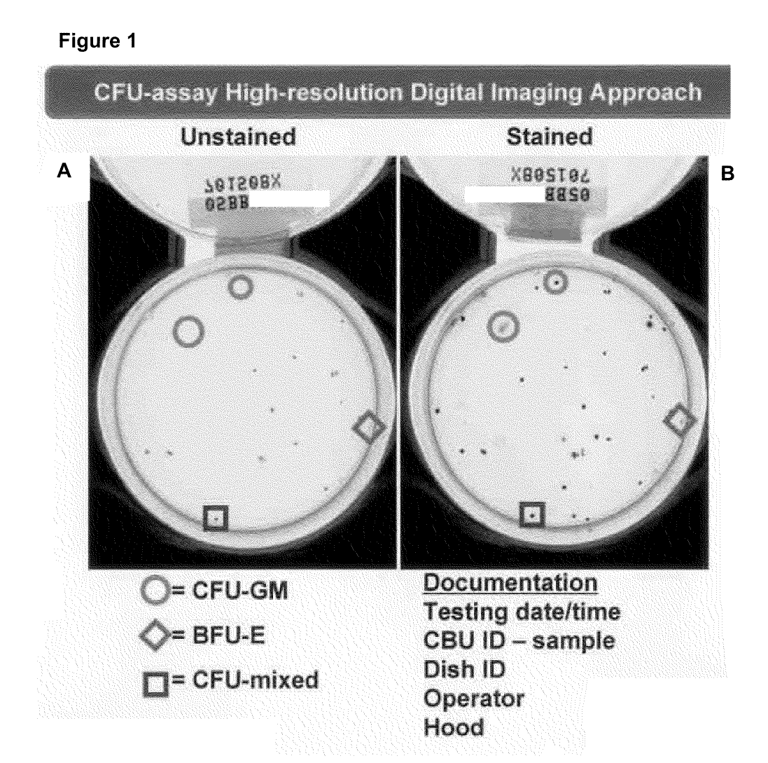

The images can be taken before and/or after staining of the culture dishes. In one embodiment the culture is imaged before and after staining. In this embodiment, the cultures can easily be classified and enumerated by comparison of unstained and stained cells and colonies if desired (see FIG. 1).

In one embodiment, a computer based laboratory information system (LIMS) is used to monitor and document each step of the CFU assay system. In one embodiment, LIMS provides the storage of culture dish images that are linked by unique barcoded ID labels of a specific CB unit. This provides positive identification of the sample linked to a specific CB unit and stores additional information such as incubator location, plating and counting dates, technician ID, as well as detailed colony enumeration (see FIG. 1).

An additional advantage of the LIMS is the portability of the stored information regarding the CB unit. That is, images and data associated with a given sample can be easily sent prior to, or with, shipment of a CB unit that is ordered for a transplant recipient. In this way, the physician of the recipient can view images of the sample and assay, and assess the quality and engraftment potential of the CB sample on site. The stored information can be sent by mail, by fax, and/or electronically. Further portability is achieved when the stored information is sent electronically.

In one embodiment, the number and functional state of the colonies can be compared with and used to estimate the engraftment potential of the cells in the sample can be determined. This is achieved using standard statistical methods standard in clinical outcome analysis, such as the Kaplan Meyer probability estimator (Kaplan, E. L. & Meier, P. (1958). “Nonparametric estimation from incomplete observations”, JOURNAL OF THE AMERICAN STATISTICAL ASSOCIATION 53: 457-481) or the cumulative incidence analysis (Gray R J (1988) A class of K-sample tests for comparing the cumulative incidence of a competing risk, ANNALS OF STATISTICS, 16:1141-1154).

To date, the 14 day CFU assay is the only assay that determines the functional state as well as the number and repopulation capacity of hematopoietic progenitor cells and is routinely performed in laboratories. Evaluation of CFU growth is manually performed by phase contrast light microscopy and CFU colonies have to be classified as CFU-GM/E, CFU-E or CFU-GM based on morphology, color and size and then, manually counted. Thus, the current CFU assay is time consuming, subjective, difficult to standardize and not practical when a large number of samples have to be tested daily. In addition, sample culture dishes cannot be stored and thus, all samples must be analyzed at the end of the culture (day 14) and there is no possibility of review, discussion, re-count and/or re-classification of colonies if needed before the dishes are discarded.

In contrast, the disclosed image detection and quantification system, assay, and methods add the advantage of processing a large number of samples/day. Images can be stored for future review and/or re-classification of colonies. Because of the specific color that each of the colonies acquires after staining, classification is objective and the enumeration of a whole dish can be quickly achieved. More importantly, the new detection system supports positive sample identification, standardization and yields high reproducibility to the assay which can be easily implemented in laboratories where large numbers of samples need to be tested daily.

In addition, the traditional CFU assay needs the use of a light inverted phase microscope to classify and enumerate CFU grown in a dish, which is subjective, and time consuming, and not practical for the enumeration of a large number of culture dishes. By using the presently disclosed system, there is no need for a microscope. Clear definition of different colony types allows a faster colony classification and enumeration and thus, helps to increase productivity. Colonies can be imaged using a variety of spectroscopic techniques, such as but not limited to, absorption and fluorescence. In this way, colony counting and classification can be standardized among different laboratories as it yields higher reproducibility than the conventional classification/enumeration methods. Images can be stored electronically for future review and counting or for physicians. The enumeration of colonies can be automated using colored images. Furthermore, the images can be easily retrieved via a barcoded ID that links them to a specific sample.

A computerized procedure allows the review of the whole CFU assay process (sample loading date and time, sample plating date and time, sample imaging date and time). Technologists who performed each step can be identified by barcoded ID labels on the image ensure positive sample identification and link to a specific sample. Additionally, the disclosed assay, method and system allow the processing of a large number of samples/day, making this a high throughput system. In one embodiment, about 100 dishes can be stained and imaged in about 2 hours. In another embodiment, about 80 dishes can be stained and imaged in less than about 2 hours. In yet another embodiment, about 60 dishes can be stained and imaged in about 70 minutes.

In another embodiment, provided herein is a computerized counting program based on current image analysis methods for identifying objects in the field of view. In one embodiment, colony boundaries are derived and improved color differentiation is achieved. This allows for a more accurate colony count and total area of each type of colony present, which can be correlated to other parameters, such as antigen markers e.g., CD34, CD38, CD79, cell number, donor demographic characteristics, and transplant outcome, drug toxicity, novel growth factors assessment, etc. Further, the assay, method and imaging and detection system provided herein can resolve current problems in experimental counting due to colonies merging or those that have burst into multiple colonies.

In one embodiment, the analysis can be expanded by locating the 2-dimensional limits of a colony in the field of vision, which enables the resolution of distinct colonies growing in close proximity and the calculation of the area occupied by each colony. The definition of the colony border also leads, by addition, to a calculation of the total number of colonies in a dish. When coupled with discriminating stains and/or cell markers defining the cell types in colonies, this approach can provide a 2- or 3-dimensional (area or volume) direct numerical estimate of the proliferation capacity of hematopoietic cells from cord blood and/or other samples, which is of a much higher accuracy than relying solely on the number of colonies. More specifically, in certain embodiments, by obtaining estimates of the third dimension (average depth) using high resolution depth-of-field measurements or by roughly assuming the average depth to be a function of the visible area and the thickness of the semi-solid growth medium layer, volume estimation is permitted. The volume of a colony yields an approximate cell number and by addition allows the estimation of overall cell numbers in all colonies in a dish or just in all colonies of a defined cell type.

Further, depending on the mechanical components utilized in the stage and optical paths, detection and spectroscopic resolution can detect colonies growing at different dimensional levels. In certain embodiments, the computerized alignment of unstained and stained images of an individual culture can be used for a time-dependent comparison between colonies during the 14-day assay procedure.

The photographic images obtained utilizing the disclosed assay and image detection and quantification system permit the computerized storage of assay results as part of a database and are useful for supporting clinical applications, including the release of cord blood units for hematopoietic transplantation.

### EXAMPLE 1

After 14 days of CB culture (CFU assay, Stem Cell Technologies), an image of a 35 mm dish was captured using a high-resolution photographic camera-based digital imaging system which achieves a resolution of 7.6 μm per pixel and thus allows a clear view of all colonies in the dish with their barcoded identifications. A short one-step staining protocol with MTT (3-[4,5-dimethylthiazol-2y1]-2,5-diphenyltetrazolium bromide) allows definition of CFU-GM/E and CFU-E (dark purple/red) and CFU-GM (light purple) by depicting each cell type in a specific color against a uniformly clear background (see FIG. 1). A good correlation was observed after comparison of the new strategy against traditional enumeration by using the microscope (R² linear=0.95; n=122 culture dishes evaluated). Low variation was observed after 151 cultures were independently classified and enumerated by three different operators (CV (coefficient variation) %=8.9%; range 1-27%) (microscope). Sample plating introduced variation in the CFU assay, in an experiment where nine CB samples were evaluated by multiple plating (intra-assay CV %=21.9%; range 3.4-34.5% and inter-assay CV %=23.3%; range 12.6-35%).

In another embodiment, a computer based laboratory information management system (LIMS) is provided to store the data related to a culture dish which is linked by a unique barcoded identification (ID) label to a specific CB unit. Additional information that can be linked to the CB unit via the barcoded ID includes the CB image, incubator location, plating and counting dates, as well as detailed colony enumeration. This system has been tested on more than 8,000 CB units in duplicate. The specific coloration of CFU colonies allows faster classification and enumeration and thus, permits a more precise analysis of CFU colonies and allows a better determination of their relationship with antigen surface markers like CD34+ cell content, drug toxicity and transplantation engraftment.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise dearly contradicted by context.

Specific embodiments disclosed herein may be further limited in the claims using “consisting of” or “consisting essentially of” language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A standardized colony forming unit assay comprising the steps of:
obtaining a sample of a hematopoietic cell-containing tissue or fluid;
culturing the cells in a medium which supports growth of hematopoietic precursor cell colonies;
staining the colonies;
imaging the colonies; and
counting the colonies for each type of precursor cell.

2. The assay of claim 1, further comprising the step of determining the functional state and engraftment potential of the colonies based upon the number and type of precursor cells in the sample.

3. The assay of claim 1 further comprising the step of isolating one or more selected from the group consisting of a subpopulation of CD34+, light density enriched cells, an apheresis product, and a combination thereof, before the culturing step.

4. The assay of claim 1 wherein said hematopoietic cell containing tissue or fluid is blood.

5. The assay of claim 1 wherein said hematopoietic cell containing tissue or fluid is cord blood.

6. The assay of claim 1 further comprising the step of saving the images in a laboratory information management system.

7. The assay of claim 1 further comprising the optional step of imaging the colonies before staining.

8. The assay of claim 1, wherein the colonies are stained in one step.

9. The assay of claim 1, wherein the stain is 3-[4,5-dimethylthiazol-2y1]-2,5-diphenyltetrazolium bromide (MTT).

10. The assay of claim 1, wherein the colonies comprise the cells selected from the group consisting of multilineage colony forming units (CFU-GM/E), granulocyte-macrophage colony forming units (CFU-GM), erythroid colony forming units (CFU-E), and combinations thereof.

11. The assay of claim 1, wherein the different precursor cell types are stained different colors.

12. An image detection and quantification system comprising:
a high resolution camera;
appropriate light alignments;
a broad spectrum flash tube; and
a positive sample identification.

13. The assay of claim 1 wherein said imaging step is performed with the image detection and quantification system of claim 12.

14. A method for determining the number of hematopoietic precursor cells in a sample comprising:
obtaining a sample of a hematopoietic cell containing tissue or fluid;
culturing the cells in a medium which supports growth of hematopoietic precursor cell colonies;
staining the colonies;
imaging the colonies; and
counting the colonies for each type of precursor cell in the sample.

15. The method of claim 14, wherein the imaging is performed with the system of claim 12.

16. The method of claim 14, wherein the hematopoietic cell containing tissue or fluid is selected from the group consisting of cord blood, peripheral blood, placenta, bone marrow, and a combination thereof.

17. The method of claim 14, wherein the colonies are selected from the group consisting of CFU-GM/E, CFU-E, CFU-GM, and a combination thereof.

18. The method of claim 14, wherein the stain is 3-[4,5-dimethylthiazol-2y1]-2,5-diphenyltetrazolium bromide (MTT).

19. The method of claim 14, further comprising the step of isolating a subpopulation of CD34+ or light density enriched cells prior to the culturing step.

20. The method of claim 14, further comprising the step of saving the images in a laboratory information management system.

21. The method of claim 14, further comprising the step of imaging the colonies prior to staining.

22. A positive sample identification comprising a sample and an identification, wherein the sample is a cord blood sample and wherein the identification identifies a specific cord blood unit and information related thereto.

23. The positive sample identification of claim 22, wherein the identification comprises a barcode associated with the cord blood sample.

24. The positive sample identification of claim 22, wherein the information related to the cord blood unit comprises one or more selected from the group consisting of the date of the sample, assay information, and combinations thereof.
